(19) United States
(12) Patent Application Publication
(10) Pub. No.: US 2008/0306346 A1
(43) Pub. Date: Dec. 11, 2008

(54) Diagnostic tool detecting the degradation status of Von Willebrand Factor multimers

(76) Inventors: Ralf A. Claus, Rothenstein (DE); Clemens Bockmeyer, Wuerzburg (DE); Hans-Peter Deigner, Lampertheim (DE); Michaela Harz, Jena (DE); Petra Roesch, Jena (DE); Juergen Popp, Jena/Kunitz (DE); Rainer Riesenberg, Jena (DE)

Correspondence Address: JORDAN AND HAMBURG LLP, 122 EAST 42ND STREET, SUITE 4000, NEW YORK, NY, 10168, US

(21) Appl. No.: 11/713,863

(22) Filed: Jun. 7, 2007

(51) Int. Cl. A61B 5/00; G01N 33/48

(52) U.S. Cl. 600/300; 356/39

(57) ABSTRACT

A method in which the cleavage profile and size distribution of von Willebrand factor (VWF) multimers is analyzed, includes: providing a sample medium of human body fluids comprising a plurality of VWF multimers of different size; enrichment or purification of the VWF multimers by cryoprecipitation or chromatography to obtain a separated preparation of the VWF multimers from said sample medium; exposing the separated preparation of VWF multimers to a light source to produce signals obtained by vibrational spectroscopy; detecting said signals; transformation by mathematical alogrithms; generation of patterns based on computing of data of original resonance spectra and determining the cleavage profile and the size distribution of said separated VWF multimers by chemometrics; and acquisition of a databank obtained from healthy individuals for identifying subjects at risk of developing at least one of the following diseases: sepsis, coagulopathy, thrombotic disease, infection, and inflammation.

### BACKGROUND OF THE INVENTION

The present invention relates to diagnostic tools such as RAMAN spectroscopy, UV-resonance Raman spectroscopy, surface enhanced Raman spectroscopy as well as Fourier transform (FT) infrared spectroscopy detecting the degradation status and molecular state of Von Willebrand Factor multimers (VWF) which plays a pivotal role in its thrombogenic activity and in the pathogenesis of thromboembolic diseases.

The present invention refers to subjects at risk of developing diseases with an altered thrombogenic activity of VWF such as inflammatory, infectious and/or coagulatory diseases.

Inflammation

Inflammation is a complex reaction of the organism to injurious agents or antigens (i.e. noxes) such as microbes and damaged—usually necrotic—cells that consist of vascular responses, migration and activation of leukocytes and systemic reactions. Protective mechanisms to combat these injurious agents lead via entrapment and phagocytosis of the offending agent by specialized cells to the unique feature of the inflammatory process: reactive response of blood vessels, accumulation of fluid and leukocytes in extravascular tissues. Local inflammation and tissue repair as one part of the host defense may be potentially harmful. However inflammatory reactions underlie common chronic diseases, such as chronic hypersensitivity reactions, atherosclerosis or lung fibrosis, as well as life-threatening acute hypersensitivity reactions or systemic inflammatory diseases such as sepsis. Thereby an uncontrolled and overwhelming immune response results in an almost lethal cascade of reactions culminating in a disease status including ischemic microcirculatory failure and multiple organ failure.

A consensus conference of the American College of Chest Physicians/Society of Critical Care Medicine in 1991 proposed standardized terminology to define various aspects of the sepsis syndrome [1]. These definitions stress the concept that the development of sepsis syndrome is related to a systemic inflammatory host response to an inciting event. The Consensus Conference recommended systemic inflammatory response syndrome (SIRS) as a general definitive term. SIRS is recognized by a constellation of cardinal signs including tachypnoea, fever or hypothermia, tachycardia, leukocytosis or leucopenia characterized by a shift to immature leukocytes in the differential white blood cell count [2]. SIRS can result from either infectious or non-infectious conditions. Non-infectious conditions which are associated with SIRS include trauma, burns, hemorrhagic or hypovolaemic shock and pancreatitis. Sepsis is defined as SIRS which results from infection, which can be of bacterial, paracystic, protozoan or viral origin.

These modern definitions of sepsis and systemic inflammatory response as outlined in the literature are forming the basis of the current invention.

Epidemiological studies from the United States of America and from Europe have shown that sepsis is a widely prevalent syndrome. Severe sepsis syndrome has important socio-economic consequences to healthcare systems as the incidence is increasing, there is significant attributed morbidity and mortality and there are substantial costs for in-hospital and post-discharge care [3].

Current therapeutically stratagems include the use of antimicrobials, focus control and aggressive physiological support, usually in an intensive care unit setting. Drotrecogin-alpha (activated) or recombinant human activated protein C (rhAPC) is the one biological agent approved for use in severe sepsis syndrome that has demonstrated efficacy in reducing 28-day all-cause mortality and new data suggests a trend towards longer term survival [4].

The pathophysiologic course of sepsis involves the release of cyto- and chemokines paralleled by the activation of endothelial and neutrophilic cells, initiating a cascade of cell-surface interactions. Activation of the coagulation system has been characterized by widespread intravascular fibrin deposition and platelet aggregation (disseminated intravascular coagulation, DIC) with subsequent microvascular and tissue injury, ultimately leading to multiple organ failure and death [5]. The contributing role of the microcirculatory dysfunction is not entirely clear, although the concentration of VWF is an important determinant for survival [6]. However, regarding the various pathogenic mechanisms that have recently been implicated in the activation of coagulation in sepsis, a reassessment of the role of the VWF degradation status is necessary.

Von Willebrand Factor

The potential role of VWF in patients with sepsis and organ failure is largely underestimated in the literature. VWF is synthesized in endothelial cells and megakaryocytes. The protein is circulating in plasma as multimers up to 20,000 kDa in size, and is essential for platelet adhesion and thrombus formation. Its deficiency or dysfunction causes an inherited bleeding disorder, von Willebrand disease [7], whereas high plasma levels are associated with an increased risk of death from severe sepsis [6].

VWF is a plasma glycoprotein required for primary haemostasis. As an extracellular adapter molecule it mediates the adhesion of platelets to subendothelial collagen of a damaged blood vessel and platelet-platelet interactions in high shear-rate conditions. The concentration of mature VWF in plasma is approximately 10 μg/mL, and its half life is about 12 hours [8,9]. VWF is synthesized in endothelial cells, where it is either secreted constitutively or stored in Weibel-Palade bodies for secretion upon stimulation, as well as in megakaryocytes, where it is stored in α-granules that later are partitioned into platelets [21]. Subsequent to the synthesis of a precursor protein, VWF undergoes a number of intracellular processing steps. Building blocks of the VWF multimer, are initially generated in a dimeric form by formation of a disulfide bond near the C-terminus. By generation of disulfide bonds near the N-termini, the protein multimerizes to a gigantic protein with a molecular mass ranging over 3 orders of magnitude to more than 20.000 kDa [7]. A single molecule may show the extraordinary length of several millimeters.

The pro-coagulant activity of VWF exhibits a non-linear function of size, since the larger the multimer, the more effective it is in promoting platelet adhesion exhibiting a critical effect on its function [10]. However, under shear stress conditions in the circulation the protein emerges more vulnerable to proteolytic digestion by limited proteolysis [11].

Regulation of VWF multimer composition in plasma is performed by two major cleaving events: first, ADAMTS13 cleaves proteolytically in between the A2 domain of each VWF monomer and second, thrombospondin-1 the disulfide bonds interlinking VWF multimers [12]. In contrast to an irreversible fragmentation of VWF by ADAMTS13, the activity of thrombospondin-1 may regulate VWF size reversibly employing a reductase activity. Thrombospondin-1 is crucially involved in the predominant VWF cleavage by ADAMTS13 due to competition with ADAMTS13 for binding to the VWF A3 domain [13].

In case of deficiency, patients have a bleeding disorder called von Willebrand disease (VWD). Occurring in up to 1% of the general population, VWD is the most common hereditary bleeding disorder, of which several subtypes are recognized. Many cases remain undiagnosed because of the mild nature of bleeding in many patients and the fact that acute phase reactions can mask the diagnosis.

Plasma concentrations of VWF protein are commonly used as an early marker for endothelial injury and dysfunction, which is almost invariably observed in systemic inflammation and infection [14]. In patients with acute lung injury and adult respiratory distress syndrome, plasma levels of VWF were found to be associated with outcome, illness severity, septic complications and the number of organ-failure free days [6]. Similar to the release of mature VWF upon stimulation by endothelial cell agonists, the plasma concentration of pro-peptide of VWF is elevated in vascular disorders, and a 4-5 fold difference in half-life of processed and unprocessed protein was observed. This raised the question whether the ratio between the proteins may serve as a tool for discrimination between chronic and acute endothelial cell perturbation [15]. In contrast to a parallel increase of both proteins e.g. in patients with diabetes, patients with acute vascular disorders such as thrombotic thrombocytopenic purpura (TTP) and sepsis exhibited a threefold elevated pro-peptide level consistent with a dramatic endothelial activation at the time of acute exacerbation. The growing body of information about the relevance of the VWF protein in inflammation indicates that the biological function is more diverse than previously thought.

ADAMTS13

The term “ADAMTS13” refers to a protein encoded by ADAMTS13, a gene responsible for familial TTP. ADAMTS13 has been identified as a unique member of the metalloproteinase gene family, ADAM (a disintegrin and metalloproteinase), whose members are membrane-anchored proteases with diverse functions. ADAMTS family members are distinguished from ADAMs by the presence of one or more thrombospondin 1-like (TSP1) domain(s) at the C-terminus and the absence of the EGF repeat, transmembrane domain and cytoplasmic tail typically observed in ADAM metalloproteinases. It is contemplated that ADAMTS13 possesses VWF (von Wildebrandt factor) cleaving protease activity.

ADAMTS13 is the main physiological modulator of the size and aggregability of VWF in plasma. In patients with thrombotic thrombocytopenic purpura (TTP), a congenital or immuno-mediated deficiency of ADAMTS13 reduces or abolishes the degradation of ultra large multimers of VWF (ULVWF) that cause the formation of intravascular platelet thrombi (thrombotic microangiopathy, TMA) [16, 17], resulting in multiorgan failure very similar to severe sepsis [18]. Determination of ADAMTS13 activity in plasma and detection of auto-inhibitors is an evident diagnostic and therapeutic marker in TMA. In other thrombotic syndromes like sepsis associated disseminated intravascular coagulation, hemolytic uremic syndrome, venoocclusive disease after bone marrow transplantation and/or drug induced thrombotic syndromes an association with diminished ADAMTS13 activity is postulated [19].

Recent data provide evidence that an altered ADAMTS13 activity and a subsequent shift in the multimeric pattern of VWF may contribute to thrombocytopenia, intravascular coagulation and microcirculatory failure in patients with severe sepsis. Nguyen and colleagues [20] reported that children with thrombocytopenia (platelet count<100,000) associated MOF had reduced or absent ADAMTS13 activity along with markedly increased plasminogen activator inhibitor-1 activity, both reversed by plasma exchange therapy. Very recently, Ono et al. described decreased ADAMTS13 levels in 109 patients with sepsis-induced DIC [21]. The incidence of acute renal failure and serum creatinine levels in patients with ADAMTS13 activity levels lower than 0.20 U/mL (incidence: 41.2%, creatinine: 1.81±1.70 mg/dL) was significantly higher than in patients with ADAMTS13 activity levels >0.20 U/mL (incidence: 15.4%, creatinine: 0.95±0.76 mg/mL) (p<0.05, p<0.01). Additionally, unusually large von Willebrand factor multimers were detected in 26 out of 51 patients (51.0%) with ADAMTS13 activity levels <0.20 U/m L.

Direct detection assays of proteolytic ADAMTS13 activity in biological samples are well known to those of ordinary skill in the art.

The present invention provides an improved method to determine the ADAMTS13 activity and the consecutive modified functional and molecular properties of VWF which ensures the efficiency and susceptibility in between the working range.

An altered ADAMTS13 activity is also postulated in connective tissue diseases like systemic lupus erythematosus or systemic sclerosis, pregnancy, bone marrow transplantation, acute and chronic inflammation, renal insufficiency and after treatment with vasoconstrictive peptide vasopressin or stabilized analogues.

Therefore detecting the degradation status and molecular state of VWF seems to play a pivotal role in monitoring diseases with an altered thrombogenic activity of VWF like inflammatory, infectious and/or coagulatory diseases.

Assessment of the functional proteolytic activity of ADAMTS13 and the detection of ULVWF may be of major clinical relevance, since plasma exchange with enzyme containing plasma preparations such as fresh frozen plasma (FFP), cryoprecipitate-poor plasma or the application of recombinant ADAMTS13 may restore the capacity to cleave ULVWF in the circulation.

VWF is suitable as a specific acute phase protein for diagnostic and therapeutic strategies because its concentration and biologic activity range significantly in dependence of the severity of the inflammatory reaction. Furthermore its half life period is adequate for detecting itself in blood, all fluids and tissue of the organism.

Till now multimeric analysis of VWF by agarose gel electrophoresis seems to be the gold standard beside nephelometric VWF ristocetin cofactor (VWF:RCo) and immunologic VWF collagen binding (VWF:CB) analysis to detect an altered binding affinity of VWF. The multimeric analysis is time consuming and the VWF:RCo as well as the VWF:CB assay have to be normalized for total VWF:Antigen concentration. Antibodies used for immunologic assays are expensive and due to cross reactions elevated false positive findings resulting in low analytic sensitivity.

An equal distribution of VWF multimers composed of 16 monomers is evident for normal ADAMTS13 activity. However, appearance of VWF multimers composed of more than 16 monomers and/or a shift to high molecular weight VWF is pathogenic for a diminished ADAMTS13 activity [22].

State of the art of VWF multimeric distribution is as follows: low molecular weight VWF (monomers one to four), intermediate molecular weight VWF (monomers five to nine) and high molecular weight VWF (more than ten monomers). Densitometric analysis verifies an equal proportional distribution of VWF multimers in normal plasma as follows: 40-50% low molecular weight VWF, 30-40% intermediate molecular weight VWF and 10-20% high molecular weight VWF [22].

An altered size of VWF multimers is intimately verified by gel electrophoresis and consecutive immunodetection. In dependence of the number of initially polymerized monomers and consecutive proteolysis by ADAMTS13 a variable number of multimer bands are detected in plasma by epitope mapping of the raveled multimers. The molecular weight of each multimer band can be verified by molecular weight markers. However, this method is highly cost intensive, time consuming and needs for detection of the antibody against VWF either radioactive chemicals, chemiluminescent chemicals or fluorophors. Illustration of high molecular weight multimers is cumbersome due to marginal separation of high molecular weight VWF multimers.

### SUMMARY OF THE INVENTION

It is an object of the present invention to provide methods to determine the degradation status of Von Willebrand Factor multimers (VWF). The invention refers to diagnostic and therapeutic monitoring of several diseases in which alteration of the degradation status of VWF plays an etiologic and/or pathogenetic role. In these diseases, like inflammatory, infectious and/or coagulatory diseases variations of the concentration and/or activity and/or molecular state of VWF are essential for prognosis, diagnosis, therapy and outcome. The methods of the present invention find use in coagulatory diseases or altering physiological states especially characterized by decreased ADAMTS13 activity and/or or pathologic platelet aggregation, as well as inflammatory diseases or altering physiological states such as systemic inflammatory response syndrome (SIRS) and/or sepsis and/or chronic inflammatory diseases.

It is an object of the present invention to provide specified methods by combination of vibrational spectroscopy and chemometrics such as UV resonance Raman spectroscopy and hierarchical cluster analysis for monitoring thrombotic and/or hemorrhagic diseases on the basis of different spectroscopic patterns.

A specifically changed Raman pattern is a biomarker for an altered molecular and functional structure of VWF and can act as a diagnostic and therapeutic indicator during the cause of diseases like inflammatory, infectious and/or coagulatory diseases. In the present invention Raman spectroscopy acts as a distinguished biomarker for differential diagnosis, severity and prognosis of disease.

The present invention provides a method of rapid identifying subjects at risk of developing or persisting a state comprising a deficiency and/or insufficient function of VWF cleaving substances like recombinant proteins with ADAMTS13 like proteolytic features; synthetic proteins with ADAMTS13 like proteolytic features; mutants, variants, fragments, and fusions of recombinant proteins with ADAMTS13 like proteolytic features; and mutants, variants, fragments, and fusions of synthetic proteins with ADAMTS13 like proteolytic features.

The present invention provides a special matrix surface for detection biological samples by vibrational spectroscopy such as quartz crystals, CaF₂ substrate, silicon carriers for Raman spectroscopy and especially KRS5, ZnSe substrates and silicon carriers FT infrared spectroscopy to improve the detection of an altered VWF structure especially ultra large VWF.

It is an object of the present invention to provide special methods for taking off whole blood, serum, plasma, tissue and other body fluids of patients developing monitoring thrombotic and/or hemorrhagic diseases such inflammatory, infectious and/or coagulatory diseases. For detection of inhibitors the withdrawal system could be filled with a physiological concentration of high molecular weight VWF and VWF cleaving products such as ADAMTS13. In dependence of inhibitors for VWF cleaving products the spectroscopic analysis results in a characteristic pattern. The withdrawal system can include every type of container. The analyzed sample could be blood collected by venous or arterial punction, sputum extracted by alveolar lavage and/or bioptic tissue. Furthermore supernatant as well as cellular components of cell cultures and/or recombinant mutants, variants, fragments, and fusions of synthetic VWF could be analyzed.

In order to restrict the number and duration of initial purification steps the present invention provides methods of VWF purification by cryoprecipitation and/or affinity chromatography. VWF is isolated from other plasma proteins such as albumin, immunoglobulin and/or fibrinogen by specific binding of VWF to the matrix of the chromatographic column. Furthermore plasmatic or soluble VWF of every description can be isolated using the affinity of VWF such as immuno-precipitation, affinity chromatography for example by columns coated with VWF antibodies, peptides like collagen (as well as mutants, variants and fragments thereof), carbohydrates like heparin (as well as mutants, variants and fragments thereof) and/or biologics of every description exhibiting binding sites towards VWF applied on a chromatographic matrix, magnetic as well as non-magnetic microparticles, and/or any other type of matrix. For separation of VWF bound VWF is eluted by adequate washing buffer. Before spectroscopic analysis microparticles and washing buffer can be eliminated as well as VWF can be resolved in an adequate medium. Finally purified VWF is in a salt free and adequate medium prepared for spectroscopic analysis.

The present invention also provides methods for recovering and purifying VWF from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxyl apatite chromatography and lectin chromatography. In other embodiments of the present invention, protein-refolding steps can be used as necessary, in completing configuration of the mature protein. In still other embodiments of the present invention, high performance liquid chromatography (HPLC) can be employed for final purification steps.

In addition, ADAMTS13 or variants or other drugs based upon this protease can also be used in several different ways. ADAMTS13 or drugs developed from it can be used in normal individuals as a novel approach to effect anticoagulation (preventing abnormal blood clots). Since blood clots are the basis of many important human diseases including heart attack and stroke, ADAMTS13 is used itself or as a suitable platform for the development of new pharmaceuticals to treat these common human diseases, where the pharmaceuticals are anticoagulants. ADAMTS13 or variants are used to deliver other therapeutic proteins specifically to the microvasculature. ADAMTS13 uses VWF in a specific conformation to cleave the Met842-Tyr843 bond. This conformation is reproduced in vitro by slightly “denaturing” VWF in urea or guanidine. It is believed that such “denaturation” is achieved in vivo by shear stress in the microvasculature. Therefore, it is contemplated that therapeutic proteins are administered in an inactive form that can be activated by cleavage of a peptide bond specifically by ADAMTS13 or variants under conditions of high shear stress in vivo. Due to this complex network an effective monitoring of drug application such as ADAMTS13 like substances is necessary. The present invention also provides methods of drug monitoring for pharmaceutical compositions used for therapy of thrombotic, hemorrhage, inflammatory and/or infectious diseases characterized by an altered ADAMTS13/VWF network. Significant changes in ADAMTS13 activity as well as the functional and molecular properties of VWF after drug application indicates an effective therapeutic efficacy of the active pharmaceutical ingredient.

The present invention provides a method detecting and monitoring this therapeutic effect.

The present invention provides a kit for analysis of the degradation status of VWF which contains the initial purification step of analyzed biological samples, the vibrational spectroscopy of the sample and the final evaluation by chemometrics. For vibrational spectroscopy RAMAN spectroscopy, UV resonance Raman spectroscopy, surface enhanced Raman spectroscopy as well as Fourier transform (FT) infrared spectroscopy can be used.

For spectroscopic analysis dried VWF multimers can be solved in agents, such as a stabilizing compound, and may be administered in any sterile, biocompatible pharmaceutical carrier, including, but not limited to, saline, buffered saline, dextrose, and water as well as any other fluid or gasiform detergent.

Analyzed samples could contain pharmaceutical compositions such as mature VWF or portions of VWF polypeptides, inhibitors or antagonists of ADAMTS13 bioactivity, including antibodies, alone or in combination. Analyzed samples can comprise pharmaceutical compositions which may bind to high molecular and/or ultralarge VWF as well as pharmaceutical compositions which may cleave VWF. Such pharmaceutical compositions can affect the molecular properties of VWF that the spectroscopic signal is enhanced.

The activity of VWF cleaving proteases such as ADAMTS13 can be measured by spectroscopic properties on the basis of a standard curve. In different biological samples of a patient ADAMTS13 activity is measured by analyzing the spectroscopic pattern. Depending on the outcome of the analysis pathogenesis of several thrombotic diseases can be evaluated for disarrangement of the degradation status of VWF. The outcome of the analysis is associated with severity, therapeutic options and outcome of the disease. Several thrombotic diseases can be analyzed such as inflammatory, infectious and/or coagulatory diseases associated with altered VWF properties.

Current treatment of diseases associated with a diminished ADAMTS13 activity consists of infusion of fresh frozen plasma with or without plasma exchange or plasmapheresis. In plasmapheresis, blood is withdrawn from the patient as for a blood donation. Then the plasma portion of the blood is removed by passing the blood through a cell separator. The cells are saved, reconstituted with a plasma substitute, and returned to the patient as a blood transfusion.

The identification of ADAMTS13 deficiency as the cause of TTP also has major implications for the treatment of this important human disease. The present invention provides methods of monitoring patients with TTP especially monitoring the administered therapeutically effective amount of a recombinant ADAMTS13 or genetic material comprising an ADAMTS13 gene or mutant or variant thereof.

Furthermore treatment comprises administering a therapeutically effective amount of ADAMTS13 protease such that the symptoms of the disease are alleviated, wherein the ADAMTS13 protease is selected from the group consisting: recombinant ADAMTS13; synthetic ADAMTS13; mutants, variants, fragments, and fusions of recombinant ADAMTS13; and mutants, variants, fragments, and fusions of synthetic ADAMTS13.

It is an object of the present invention to provide methods to determine the susceptibility of individuals to these treatments of disease, in efforts to prevent the appearance and/or severity of symptoms. What is also needed is a method to identify those individuals for whom the disease appears to be genetic, to monitor the efficiency of drug therapy such as application of ADAMTS13 like drugs or drugs influencing the ADAMTS13 activity and to monitor therapeutic strategies in general.

The methods of the present invention find use in monitoring the treatment of diseases or altering physiological states characterized by decreased VWF-cleaving protease activity, and/or pathologic platelet aggregation. The invention provides methods for monitoring the increasing VWF-cleaving protease activity and/or decreasing pathologic platelet aggregation by administering peptides or peptide fragments or variants of ADAMTS13. Alternatively, drugs which act to increase VWF-cleaving protease activity and/or decreasing pathologic platelet aggregation are monitored through screening methods described above.

The present invention provides methods for monitoring of the interaction between VWF and other coagulation factors especially Factor VIII. Factor VIII and Factor VIIIa are protected from proteolytic inactivation by activated protein C in presence of VWF. Therefore monitoring the affinity of VWF for Factor VIII plays a pivotal role during therapy of sepsis especially septic shock by activated protein C due to monitoring the efficacy of activated protein C treatment.

Due to the high sensitivity and specificity of the methods described above the present invention provides new therapeutic aspects by the opportunity of monitoring administered pharmaceuticals with a sensitive and specific VWF-cleaving protease activity.

It is an object of the present invention to provide a one step method brigading sample preparation, spectroscopical analysis and completing chemometrics.

It is an object of the present invention to provide an assay kit for detection of an altered VWF cleavage pattern including a reference sample with physiological ADAMTS13 activity and VWF cleavage pattern as well as a reference sample with an altered physiological ADAMTS13 activity and VWF cleavage pattern. Furthermore the kit can include reagents like washing buffer, dilution buffer and reagents for accumulation of VWF.

It is an object of the present invention to provide an multifunctional assay kit for detection of VWF:Ag, VWF multimers, VWF:CB, VWF:Rco and/or VWF:FVIII by calibration of these conventional assays with the spectroscopical analysis provided in the present invention.

It is an object of the present invention to provide a highly sensitive and specific method for screening individuals at risk developing and/or presenting an altered VWF cleavage pattern without any clinical symptoms.

It is an object of the present invention to provide a new diagnostic parameter without any clinical indication but providing a screening parameter for developing any kind of disease.

In particular the present invention relates to thrombotic diseases, hemorrhage diseases, inflammatory diseases especially systemic inflammatory diseases such as sepsis or tumortoxic diseases and/or infectious diseases caused by bacteria, viruses, parasites and/or fungi. Furthermore the present invention relates to any kind of disease associated with an altered VWF cleavage pattern like burn disease, tissue damage cuased by surgery or trauma, obstetric complication like HELLP syndrome, organ and/or bone marrow transplantation, thrombocytopenia associated diseases like M. Werlhoff, chronic inflammatory disease like chronic bowel disease, sklerodermia, systemic lupus erythematodes, rheumatoid arthritis, vasculitis or any kind of autoimmune disease. Furthermore metabolic diseases like Diabetes mellitus, endocrine diseases, atherosclerosis and or any kind of organ insufficiency due to microcirculatory failure.

The present invention relates to any kind of disease associated with an endothelial dysfunction which are well known to those of ordinary skill in the art.

The present invention provides methodological and laboratory approaches to detect and describe an altered biofunctionality of VWF for a more effective and specific diagnostic and therapeutic monitoring of inflammatory, infectious and/or coagulatory diseases.

It is an object of the present invention to provide spectroscopic methods such as Raman spectroscopy, UV resonance Raman spectroscopy; surface enhanced Raman spectroscopy as well as FT infrared spectroscopy for detecting the degradation status of VWF in blood samples, tissue and other probes and body fluids of the organism. Resoluble cryoprecipitate and affinity chromatographic fractions of plasma from patients with coagulatory diseases like thrombotic microangiopathy containing VWF represent a specific spectrum with diagnostic and therapeutic significance towards healthy controls.

Raman Spectroscopy

Raman spectroscopy provides information about the vibrational state of molecules. On a molecular level, molecules consist of atomic bonds capable of existing in a distinct number of vibrational states. If there is an incident radiation the molecule is excited in a virtual eigenstate. Because this virtual level does not correspond to a real energy level of the molecule, it has to decay very fast by radiation of elastic or inelastic scattered light.

Most often, the scattered light has the same wavelength as the incident light, a process designated Rayleigh or elastic scattering. In some instances, the irridated radiation can contain slightly more or slightly less energy than the incident radiation which is depending on the allowable vibrational states and the initial and final vibrational states of the molecule. The difference in energy is consumed by a transition between allowable vibrational states, and these vibrational transitions exhibit characteristic values for particular chemical bonds, which accounts for the specificity of vibrational spectroscopic technologies such as Raman spectroscopy.

The result of the energy difference between the incident and scattered radiation is manifested as a shift in the wavelength between the incident and re-radiated radiation, and the degree of difference is designated the Raman shift (RS), measured in units of wavenumber (cm⁻¹). If the incident light is substantially monochromatic (single wavelength) as it is when using a laser source, the inelastic Raman scattered light which differs in frequency can be more easily distinguished from the Rayleigh scattered light.

Because Raman spectroscopy is based on irradiation on a sample and detection of its scattered light, it can be employed non-invasively and non-destructively, such that it is suitable for analysis of biological samples in situ. Water exhibits scarcely Raman scattering (e.g., water exhibits significantly less Raman scattering than infrared absorbance), and Raman spectroscopy techniques can be readily performed in aqueous environments. Raman spectral analysis can be used to assess occurrence of and to quantify blood components and components of other tissues.

The Raman spectrum of a compound or a mixture of compounds can reveal the molecular composition of that material, including the specific functional groups present in organic and inorganic molecules. Raman spectroscopy is useful for detection of metabolites, pathogens, and pharmaceutical and other chemical agents because every molecule exhibit characteristic ‘fingerprint’ Raman spectra, subject to various selection rules, by which the agent can be identified. Peak position as well as peak shape of Raman spectra, and adherence to selection rules can be used to determine molecular (or cell) identity.

In the past several years, a number of key technologies have been introduced into wide use that has enabled scientists to largely overcome the problems inherent to Raman spectroscopy. These technologies include high efficiency solid-state, gas or semiconductor lasers, interference filters or gratings to remove side bands from the laser light, efficient laser rejection filters and silicon CCD detectors. In general, the wavelength and bandwidth of light used to illuminate the sample is not critical, so long as the other optical elements of the system operate in the same spectral range as the light source.

In order to detect Raman scattered light and to accurately determine the Raman shift of that light, the sample should be irradiated with substantially monochromatic light, such as light having a bandwidth <1.3 nanometers, and preferably not upgrading 1.0, 0.50, or 0.25 nanometer. Suitable sources include various combinations of laser generators and polychromatic light source-monochromators. It is recognized that the bandwidth of the irradiating light, the resolution of the wavelength resolving element(s), and the spectral range of the detector determine how well a spectral feature can be observed, detected, or distinguished from other spectral features. The combined properties of these elements (i.e., the light source, filter, grating, or other mechanism used to distinguish Raman scattered light by wavelength) define the spectral resolution of the Raman signal detection system. The known relationships of these elements enable the skilled artisan to select appropriate components in readily calculable ways. Limitations in spectral resolution of the system (e.g., limitations relating to the bandwidth of irradiating light, grating groove density, slit width, interferometer stepping, and other factors) can limit the ability to resolve, detect, or distinguish spectral features. An expert in the field understands that and how the separation and shape of Raman scattering signals can determine the acceptable limits of spectral resolution for the system for any of the Raman spectral features described herein.

Typically, a Raman peak that both is distinctive of the substance of interest and exhibits an acceptable signal-to-noise ratio will be selected. Multiple Raman shift values characteristic of the substance can be assessed, as can the shape of a Raman spectral region that may include multiple Raman peaks. If the sample includes unknown components, then the entire Raman spectrum can be scanned during spectral data acquisition, so that the contributions of any contaminants to the data can be assessed.

Normal Raman Scattering (Non-Resonance Raman); Inelastic Light Scattering, Light Scattered at v−vo (Stokes) or v+vo (Anti Stokes)

Resonance Raman; Similar to the Non-resonance Raman, only difference is scattered light is high enough to excite to the higher electronic states which corresponds normally to UV region. And inelastic scattering of the light; Resonance Raman spectroscopic intensity is 10⁸ times higher than normal Raman. That's why the Raman scattering is new tool for studying biological systems. Normal Raman specs' intensity was bad for studying amide bonds.

UV-Resonance Raman Spectroscopy for Studying Protein Folding

UV-resonance Raman spectroscopy is a wide tool for studying protein conformation, folding and unfolding, even early stages of protein folding (ns or less) and size of multimerized proteins can be studied.

Surface Enhanced Raman Spectroscopy

The use of Raman Scattering to investigate adsorbates on surfaces was initially thought to be of insufficient sensitivity. However, it was discovered that certain molecules and appropriately prepared metal surfaces could display Raman scattering cross-sections many orders of magnitude greater than for isolated molecules. Raman Scattering is carried out using infra red light.

SERS is used to investigate the vibrational properties of adsorbed molecules. Metal surfaces have to be of high reflectivity and of a suitable roughness. Increasing sensitivity of detectors these days means that Raman spectra can be observed in very thin films without the need for the surface enhancement effect. (http://www.uksaf.org/tech/sers.html)

FT Infrared Spectroscopy

For detection of FT infrared spectra a Michelson Interferometer for use on an optical table is necessary. The Michelson interferometer is the most common configuration for optical interferometry. An interference pattern is produced by splitting a beam of light into two paths, bouncing the beams back and recombining them. The different paths may be of different lengths or be comprised of different materials to create alternating interference fringes on a back detector.

Michelson Interferometer

There are two paths from the (light) source to the detector. One reflects off the semi-transparent mirror, goes to the top mirror and then reflects back, goes through the semi-transparent mirror, to the detector. The other first goes through the semi-transparent mirror, to the mirror on the right, reflects back to the semi-transparent mirror, then reflects from the semi-transparent mirror into the detector.

If these two paths differ by a whole number (including 0) of wavelengths, there is constructive interference and a strong signal at the detector. If they differ by a whole number and a half wavelength (e.g., 0.5, 1.5, 2.5 . . . ) there is destructive interference and a weak signal. This might appear at first sight to violate conservation of energy. However energy is conserved, because there is a re-distribution of energy at the detector in which the energy at the destructive sites are re-distributed to the constructive sites. The effect of the interference is to alter the share of the reflected light which heads for the detector and the remainder which heads back in the direction of the source. The Michelson Interferometer has been used for the detection of gravitational waves, as a tunable narrow band filter, and as the core of Fourier transform spectroscopy.

Cryoprecipitated blood samples from patients with thrombotic microangiopathy vs. healthy controls were analyzed by different types of spectroscopy. For Raman spectroscopy samples were irradiated with substantially monochromatic light having a laser wavelength of 244 nm. UV-resonance Raman spectroscopy is well established for functional characterization of macromolecules such as proteins due to selective irradiation of macromolecules. Proteins are main content of blood plasma. Therefore low concentration of proteins is sufficient for UV-resonance Raman spectroscopically detection and plasma is representative for detection of proteins without any further purification except cryoprecipitation. Cryoprecipitation is the method of choice for enrichment of plasma VWF. Although Raman spectra of analyzed plasma samples are very similar at first glance, the complex architecture of vibrational spectroscopy contains important information. A characteristic ‘fingerprint’ Raman spectra about 1800 cm⁻¹ contain main vibrational peaks for analysis of VWF. Raman spectrum of blood plasma resulted of vibration from aromatic amino acids such as tryptophan, tyrosine and phenylalanine as well as amids I, II and III.

Raman spectra of interest between patients and healthy controls are cumbersome to differentiate due to the complexity of obtained spectra. A systematic way to handle and analyze Raman spectra is needed to effectively extract relevant information. Raman spectra can be analyzed by chemometric methods. Chemometric methods itself can be defined as the application of mathematical, statistical, graphical or symbolic methods to maximize the chemical information which can be extracted from data. Chemometric procedures can prove useful at any point in an analysis, from the first conception of an experiment, until the data is discarded. Chemometric methods are widely used in order to plan, develop, analyze and validate methods and experiments especially for a large and related panel of data. Therefore a chemometric approach can be used for monitoring of biological samples analyzed by Raman spectroscopy.

Pattern recognition approaches seek to identify similarities and regularities present in data. A major subset of pattern recognition is cluster analysis. Cluster analysis seeks to perceive natural classifications, often called clusters, in data. Pattern recognition and cluster analysis problems are usually trivial in two or three dimensions, since people are excellent at such discriminations when they can plot the data. When more dimensions are involved, computers are usually used to assist.

Due to the huge amount of data obtained by use of multi-channel techniques such as infrared and Raman spectroscopy, the full potential for these techniques to adequately analyze differences and similarities within and between sets of samples or to discriminate between samples or obtain analyte concentrations in the presence of uncharacterized and varying matrices can only be achieved by applying appropriate statistical techniques, which are often collectively referred to as chemometrics. In the context of the present invention, the used chemometric technique has been assigned to the area of cluster analysis and pattern recognition which seek to identify regularities and similarities present in the data. Among these are direct two and three dimensional plots, projection and mapping, cluster and discriminant analysis. The K-nearest neighbor's analysis is a technique predicting group membership of a sample based on the group membership of its K nearest neighbors. This procedure involves calculating the distance between each pair of points and choosing one or several values of K. The group identities of each of the K nearest neighbors for each of the samples of interest are tallied. The group with the largest number of “votes” for each sample is the group that that sample is assigned to. In this analysis, all of the cases were considered to be samples of interest. Linear discriminant analysis is one of a family of techniques which seek to find hyperplanes, planes in n-dimensional space, which separate one category from another. Some of these techniques are iterative, seeking to use as few variables as possible. Support vector machines (SVM) are effective algorithms for modeling multivariate, non-linear systems requiring the lowest number of calibration samples to achieve superior predictive performance [Cogdill R P, Dardenne P, J Near infrared spectroscopy (2004) 12, 93-100].

For validation it is necessary to generate a comprehensive set of Raman spectra of a blood bank of patients vs. healthy controls, whereas these data are utilized to establish a data base. A chemometric model is established in order to differentiate between patients vs. healthy controls. The completed data base of spectra is used to detect an unknown sample by comparison of the analyzed spectra and stored spectra of the data base.

The present invention provides a method of detecting the presence or absence of ULVWF as well as variants of the VWF molecular structure and molecular weight and function, furthermore indirect detecting agents that regulate molecular weight and function of VWF such as ADAMTS13.

The present invention provides an improved method for diagnostic and therapeutic monitoring of several thrombotic and microthrombotic as well as hemorrhage diseases. The present invention provides an improved method to decrease fatality and the appearance and/or severity of the subsequent debilitating symptoms associated with these diseases. The molecular weight, function and structure of VWF are particularly related to one spectroscopic pattern. The present invention provides an improved method to determine the susceptibility of individuals to the disease, in effort to prevent the appearance and/or severity of symptoms and to identify those individuals for whom the disease appears to be genetic.

Therefore the present invention enables to evaluate the molecular weight, function and structure of VWF as well as indirect detecting of regulating agents in biological samples such as whole blood, serum, plasma, tissue and other body fluids for diagnostic and therapeutic strategies. Detection of spectra which are characteristic for high molecular and/or ultra large VWF is evident for a diminished proteolysis of VWF.

Disease monitoring by Raman spectroscopy offers an effective, causal treatment as well as validation of the therapeutic efficacy. A patient sample with an insufficient proteolytic activity for VWF is well defined differentiated from a healthy donor by Raman spectroscopy.

The present invention provides methods for using the degradation status of VWF for screening drugs that can alter and/or induce the proteolytic activity for VWF.

The present invention provides methods for a rapid detection and specific molecular characterization of thrombotic diseases.

### BRIEF DESCRIPTION OF THE DRAWINGS

FIGS. 1A, 1B, 2, 3A, 3B, 4A and 4B are UV resonance RAMAN spectra of human plasma samples;

FIG. 5 is a dendrogram resulting from hierarchical cluster analysis of human plasma sample spectra;

FIGS. 6A, 6B, 7A and 7B are FT-IR spectra of human plasma samples; and

FIG. 8 is a dendrogram resulting from hierarchical cluster analysis of human plasma sample spectra.

### DETAILED DESCRIPTION OF THE INVENTION

Methods

Citrated plasma specimens for measurement of VWF-Antigen (VWF:Ag), VWF multimer analysis and ADAMTS13 activity were stored at −80° C. for later assaying.

VWF:Ag was measured by an enzyme-linked immuno-sorbent assay, using polyclonal anti-VWF antibody (Dako, Hamburg, Germany).

ADAMTS13 activity was determined by the collagen binding method using recombinant human VWF as substrate [23, 24]. All samples were tested at dilutions 1:10, 1:20 and 1:40, and mean values were calculated. Intra- and inter-assay coefficients of variation were 12% on the basis of six replicates and 16% in six different runs, respectively. One Unit/mL (U/mL) ADAMTS13 activity is defined as the proteolytic activity of one mL pooled normal human plasma from 45 healthy individuals presenting 100% activity. The detection limit of the assay was 0.05 U/mL. The specificity of the assay was verified by dilution experiments with plasma from TTP patients containing autoantibodies [23]. By dilution experiments with normal plasma the presence of autoantibodies was excluded in all patient samples.

Multimer analysis of VWF was carried out by agarose gelelectrophoresis (60 V, 16° C., 15 h) in a Multiphor (Amersham Pharmacia Biotech, Freiburg, Germany) using 1.2% LGT-Agarose (Sigma-Aldrich, Seelze, Germany). After blotting on nitrocellulose membranes (33V, 2.5 mA, 2 h) luminescent visualisation was performed using HRP-VWF-Ab (Dako Hamburg, Germany) and ECL-Detection kit from BioRad [25-27]. Plasma samples were normalized to an equal amount VWF:Ag (0.1-0.05 U/mL). For comparison, VWF multimers of a normal plasma pool (NPP; n=40) are shown. Illustrations of all gels in the present description are without any use of non-linear adjustment. The amount of high molecular weight multimers in NPP was defined as 100% and compared to those of patients.

Preanalytical sample preparation: 200 μl citrated plasma from healthy controls as well as from patients with thrombotic microangiopathy of different origin diagnosed by a residual proteolytic activity of ADAMTS13<20% as determined by the method of Gerritsen [24] was cryoprecipitated by thawing on ice for 45 min after freezing at −80° C. for about 6 h. Afterward the samples were centrifugated at 18,000 g for 30 min at 4° C. The supernatant was discarded and the cryoprecipitated pellet was resolubilized in a total volume of 20 to 30 μL phosphate-buffered saline (PBS) solution. For UV-Raman analysis, the cryoprecipitated protein mixture was spotted onto fused silica plates and dried in vacuo.

The plasma components were purchased by Sigma-Aldrich (Taufkirchen, Germany). Recombinant factor VIII was purchased from Baxter (Wien). The substances were diluted in water or PBS in appropriate concentrations and spotted onto fused silica plates and dried in vacuo. For comparison of VWF multimers differing in length and thrombophilic activity, we used two different VWF specimens: (i) ultralarge VWF was obtained from recombinant protein synthesis 20 and (ii) low-molecular-weight VWF was obtained by limited proteolysis of rhVWF by ADAMTS13 at mild denaturating conditions (1.5 M urea) in the presence of Ba²⁺ over 2 h as described [23].

Spectroscopic instrumentation: The UVRR data were collected on a micro-Raman instrument (HR800, Horiba/Jobin Yvon) equipped with a 2400 groove mm⁻¹ grating and a cryogenically cooled CCD detector. An intracavity frequency doubled argon ion laser (Innova 300, FReD, Coherent) provided the 243.993 nm continuous wave laser lines. Approximately 1 mW was delivered to the sample. The wavenumber accuracy of the HR800 spectrometer is ±4 cm⁻¹. Incident light on the sample and 180° backscattered light was collected by a broadband anti-reflection coated UV micro spot objective (LMU UVB, 40×/0.50) with a working distance of 1 mm. Photochemical decomposition was limited by rotating the blood plasma samples at 6 rpm on a turning knob, whereby the turning knob has been moved in the xy-direction after each turn. A video camera, which is sensitive in the UV and in the visible spectral range, was used for positioning of the samples under the microscope. The spectrometers entrance hole was set to 300 μm. An accumulation time of 120 s-240 s was chosen for each spectrum.

The UVRR data were collected on a micro-Raman instrument (HR800, Horiba/Jobin Yvon) equipped with a 2400 groove mm⁻¹ grating and a cryogenically cooled CCD detector. An intracavity frequency doubled argon ion laser AQ6 (Innova 300, FReD, Coherent) provided the 243.993 nm continuous wave laser beam. Approximately 1 mW was delivered to the sample. The wavenumber accuracy of the HR800 spectrometer is 4 cm⁻¹. Incident light on the sample and 180° backscattered light was collected by a broadband antireflection-coated UV microspot objective (LMU UVB, 40×/0.50) with a working distance of 1 mm. Photochemical decomposition was limited by rotating the blood plasma samples at 6 rpm on a turning knob, whereby the turning knob was moved in the xy-direction after each turn. A video camera, which is sensitive in the UV and the visible spectral range, was used for positioning of the samples under the microscope. The spectrometer's entrance hole was set to 300 μm. An accumulation time of 120-240 s was chosen for each spectrum.

An unsupervised classification chemometric method, the hierarchical cluster analysis, was applied to differentiate between cryoprecipitated plasma samples of healthy controls and patients with TMA, which was performed by the use of the program OPUS IDENT from Bruker.

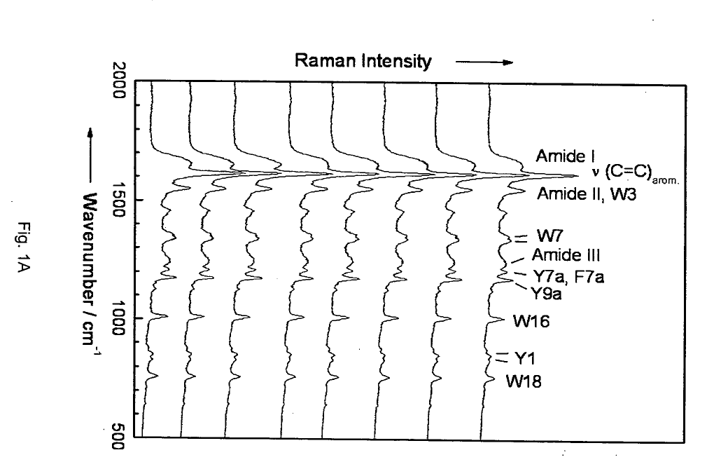

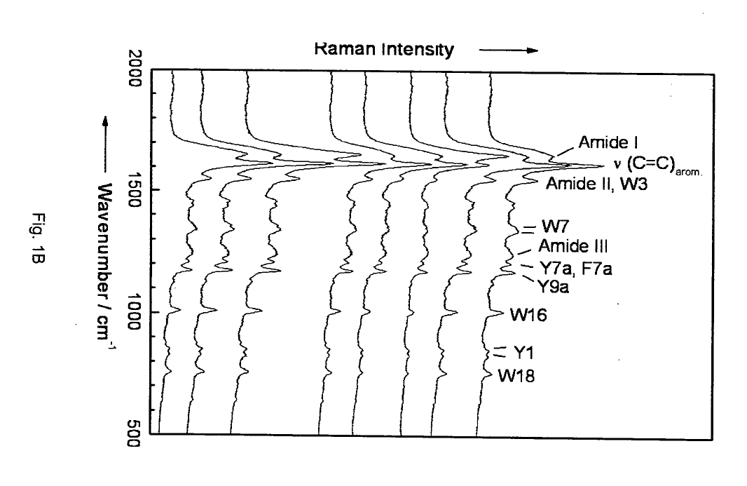

In FIGS. 1(A) and 1(B), various UV-resonance Raman spectra of plasma samples of healthy donors (FIG. 1(A)) and patients with thrombotic microangiopathy (FIG. 1(B)) are represented, illustrating considerable variations in the absolute and relative intensities of the bands of 1551, 1615, and 1650 cm⁻¹ between patients with TMA and healthy donors. Differences of the absolute intensities are due to the inhomogeneous distribution and variations in thickness resulting from surface tension across the plasma spot. Variation in the background intensities can be attributed to mild pyrolysis of the sample despite moving the sample during measurement.

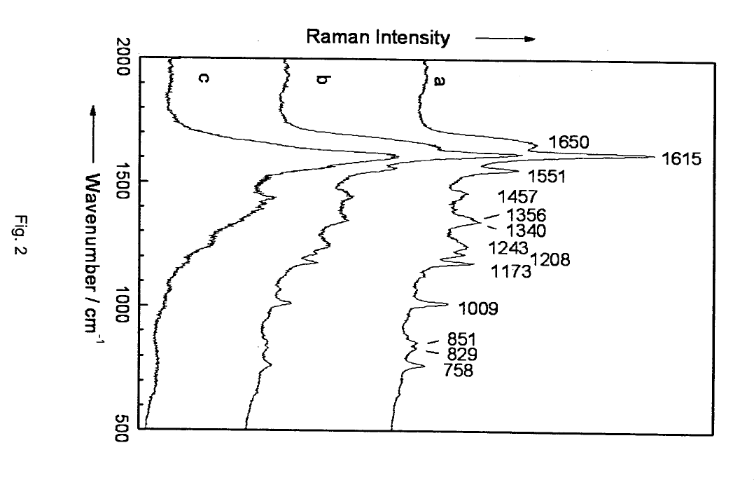

FIG. 2 illustrates the influence of photochemical degradation of the plasma samples during measurement with an accumulation time of 2 min. When the sample was rotated and moved in the xy-direction after each turn during spectrum recording, the best resolution of the spectrum was demonstrated compared with that obtained when the sample was rotated and not moved in the xy-direction (FIG. 2b) and when the sample was measured by keeping the laser beam position fixed on the sample (FIG. 2c). Therefore the samples were rotated and moved during measurement to minimize the photochemical decomposition of the plasma samples. Additionally, there is a significant change in the relative intensities of the two principal bands at 1615 and 1650 cm⁻¹ for healthy donors. Most of the samples show an increased intensity at 1615 cm⁻¹ compared with the signal at 1650 cm⁻¹. The signal at 1551 cm⁻¹ is due to vibrations of tryptophan and the amide II vibration. The amide II vibration reflects the N—H bending coupled with the C—N stretching mode. The signal at 1615 cm⁻¹ can be attributed to in-plane ring stretching vibrations of aromatic amino acids. The band at 1650 cm⁻¹ can be assigned to the amide I vibration, the C—O stretching, and N—H in-plane bending vibration, and the amino acid phenylalanine. The amide III mode is located at 1243 cm⁻¹ and results from the N—H and C—C-vibration.

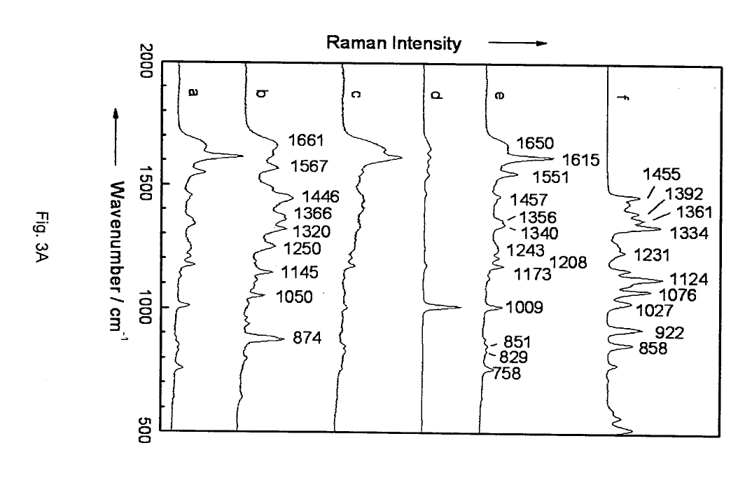

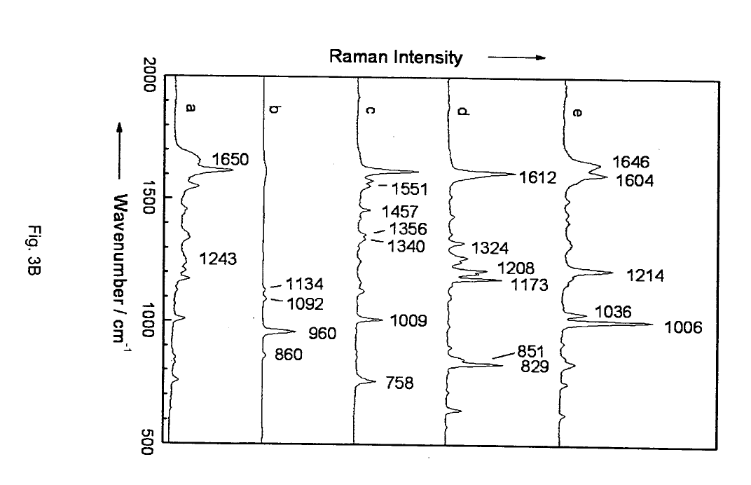

To characterize cryoprecipitated human plasma and to elucidate the differences of the relative intensity of the bands at 1615 and 1650 cm⁻¹ for healthy controls, different plasma components such as high abundance proteins were investigated. FIG. 3A represents UV-resonance Raman spectra of a plasma sample of a healthy donor (a) opposed to clotting factor VIII (b), ultra large VWF multimers (c), proteolyzed VWF (d), fibrinogen (e), and glucose (f). Glucose exhibits various bands. The two prominent bands at 1334 and 1124 cm⁻¹ can contribute slightly to the plasma spectrum. The spectrum of fibrinogen (e) reveals nearly the same bands as a plasma sample featuring some differences in the relative intensities of the three bands at 1551, 1615, and 1650 cm⁻¹. Furthermore proteolyzed VWF fragments (d) that give an intense band at 1009 cm⁻¹ and some small signals at 1176, 1543, 1580, 1616, and 1650 cm⁻¹ were analyzed. Ultra large VWF multimers (c) were investigated that are present in a complex with factor VIII in patients' plasma samples. This spectrum looks also similar to that of the plasma sample, although the bands are less intense. The spectrum of clotting factor VIII (b) shows two prominent bands at 874 and 1446 cm⁻¹ and various weaker peaks at 1145, 1250, 1320, 1366, 1567, and 1567 cm⁻¹. In FIG. 3B, UV-resonance Raman spectra of blood plasma of a healthy donor (a), PBS (b), tryptophan (c), tyrosine (d), and phenylalanine (e) are illustrated. The plasma samples were resolubilized in PBS, hence PBS was measured to exclude distortion arising from the buffer. The spectrum of PBS (b) shows an intense signal at 960 cm⁻¹ and some small bands at 860, 1092, and 1134 cm⁻¹. These bands are not detectable in the human plasma spectrum, excluding an attributable role of PBS in the assay system. Since using UV-resonance Raman spectroscopy aromatic amino acids are discriminatory enhanced, three important amino acids, tryptophan, tyrosine, and phenylalanine, were analyzed. Tryptophan (c) exhibits characteristic bands at 758 and 1009 cm⁻¹ due to symmetric benzene/pyrrole in-phase and out-of-phase breathing modes. The signals at 1340 and 1356 cm⁻¹ can be attributed to the vibration resulting from the fermi resonance between the N1-C8 stretching in the pyrrole ring and combination bands of the out-of plane bending. The signal of the C—C stretching vibration of the pyrrole ring is located at 1551 cm⁻¹. The C—C stretching mode of all aromatic acids gives a band at 1615 cm⁻¹. The symmetric ring stretching mode of tyrosine (d) is located with an intense band at 829 cm⁻¹ connected with a shoulder at 851 cm⁻¹. The signal at 1173 cm⁻¹ can be assigned to the in-plane C—H bending vibration. The band at 1208 cm⁻¹ can be attributed to the ring C—C-stretching mode of tyrosine and phenylalanine (e). The signal of tyrosine at 1615 and of phenylalanine at 1604 cm⁻¹ is due to the in-plane ring stretching vibration. An additional band of phenylalanine is seen for the ring breathing mode at 1006 cm⁻¹. Comparing the signals of plasma components with those of cryoprecipitated human plasma, the most common peaks arise from the amino acids tryptophan, tyrosine, and phenylalanine.

FIG. 3A shows UV resonance Raman spectra of a human plasma sample of a healthy donor (a), recombinant factor VIII (b), ultralarge VWF multimers (c), proteolyzed VWF fragments (d), fibrinogen (e) and glucose (f).

FIG. 3B shows UV resonance Raman spectra of blood plasma of a healthy donor (a), phosphate buffered saline (b), tryptophan (c), tyrosine (d) and phenylalanine (e).

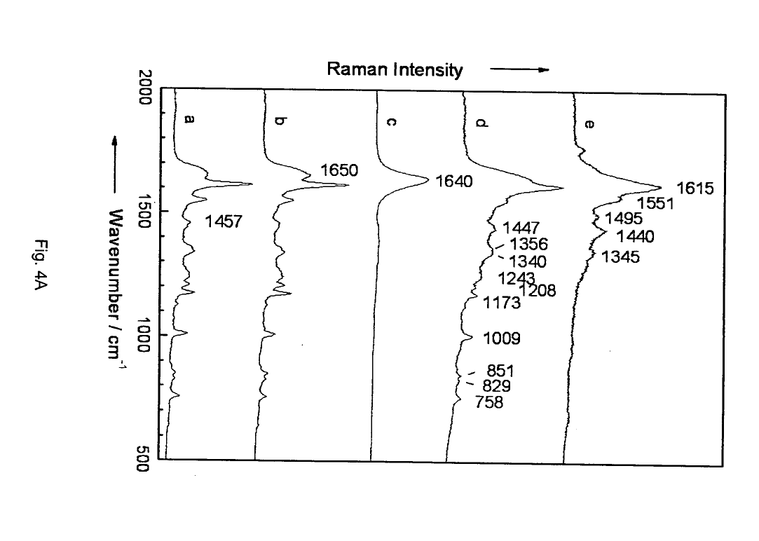

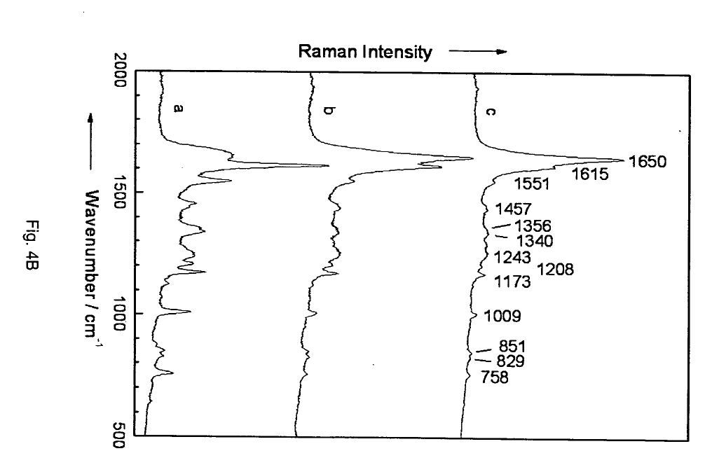

Some plasma samples differed in intensity of yellowness. This effect could be caused by different endogenous dyes. Therefore β-carotene was studied because it is often responsible for pigmentation in biological samples such as human plasma. Furthermore whole blood was measured to investigate whether these variations were caused by other components of whole blood, such as cellular components. The dye hemoglobin from erythrocytes and its degradation product bilirubin were also investigated. FIG. 4A illustrates UV-resonance Raman spectra of a human plasma sample of a healthy donor (a), whole blood (b), β-carotene (c), hemoglobin (d), and bilirubin (e). The spectra of whole blood, hemoglobin, and bilirubin do not reflect an increased band at 1650 cm⁻¹ as seen in some plasma samples. These components show an intense band at 1615 cm⁻¹. Hence these variations of the relative intensities between the 1615 and 1650 cm⁻¹ peaks do not occur because of the availability of some different blood components in the plasma sample. β-carotene shows one dominant broad band at 1640 cm⁻¹ (c). This signal does not occur at the spectrum of human plasma. Thus β-carotene does not contribute decisively to the human plasma spectra; it only contributed to the peak at 1650 cm⁻¹ with a slight shoulder. In addition to the various dyes, high-density lipoprotein from human plasma as a lead structure for human lipoproteins was investigated to clarify the differences in the relative intensities between the 1615 and 1650 cm⁻¹ peaks. In FIG. 4B, UV-resonance Raman spectra of blood plasma of a healthy donor showing increased intensity at 1615 cm⁻¹ compared with the signal at 1650 cm⁻¹ (a), a human plasma sample of a healthy donor showing decreased intensity at 1615 cm⁻¹ relative to the signal at 1650 cm⁻¹ (b), and lipoprotein (c) are represented. Lipoproteins exhibit an increased intensity at 1650 cm⁻¹ compared with the signal at 1615 cm⁻¹. Therefore the plasma samples with the raised band at 1650 cm⁻¹ offer a high content of lipoproteins. Normally lipids should not be present with high concentrations in the cryoprecipitated plasma. Plasma sample spectra with an absence of the increased band at 1650 cm⁻¹ may serve as a method for quality control of sample preparation. To classify the analyzed plasma samples of healthy donors and patients with thrombotic microangiopathy, an unsupervised method, the hierarchical cluster analysis, was performed. Only spectra without an increased peak at 1650 cm⁻¹ were used for classification. The spectra were pretreated by vector normalization and the spectral range between 600 and 1800 cm⁻¹ was chosen for classification. The spectral distances between each spectrum were calculated with the standard method. Ward's technique was used to calculate the spectral distances between a newly created cluster and all of the other spectra or identified clusters.

FIG. 4A shows UV resonance Raman spectra of a human plasma sample of a healthy donor (a), whole blood (b), β-carotene (c), hemoglobin (d) and bilirubin (e).

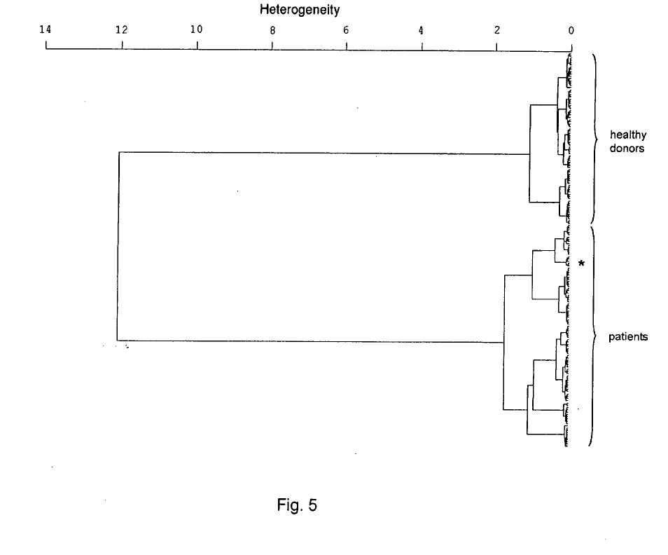

FIG. 4B shows UV resonance Raman spectra of a blood plasma of a healthy donor showing an increased intensity at 1615 cm⁻¹ compared to the signal of 1650 cm⁻¹ (a), of a human plasma samples of a healthy donor showing an decreased intensity at 1615 cm⁻¹ relative to the signal of 1650 cm⁻¹ (b) and high density lipoprotein from human plasma (c). FIG. 5 shows the dendrogram of the resultant classification of the cryoprecipitated plasma samples based on 175 spectra of 8 healthy controls and 10 different patients' samples. The smaller the spectral distances in the dendrogram the more similar are the spectra. The dendrogram shows a clear separation of healthy controls and patients; however the spectrum of one healthy control was falsely classified to the patients' cluster for unknown reasons. This spectrum is indicated in the figure by an asterisk.

The dendogram of FIG. 5 results from hierarchical cluster analysis of plasma sample spectra of the healthy controls and patients with thrombotic microangiopathy based on the spectral range of 600-1800 cm⁻¹.

Similar investigations were performed by means of FT-IR spectroscopy. FT-IR spectra were recorded with a FT-IR spectrometer (IFS66, Bruker) in the spectral region of 400 and 6000 cm−1 with a resolution of 4 cm⁻¹. As a radiation source a globar was used as well as a DTGS-detector (deuterated triglycine sulfate) for detection.

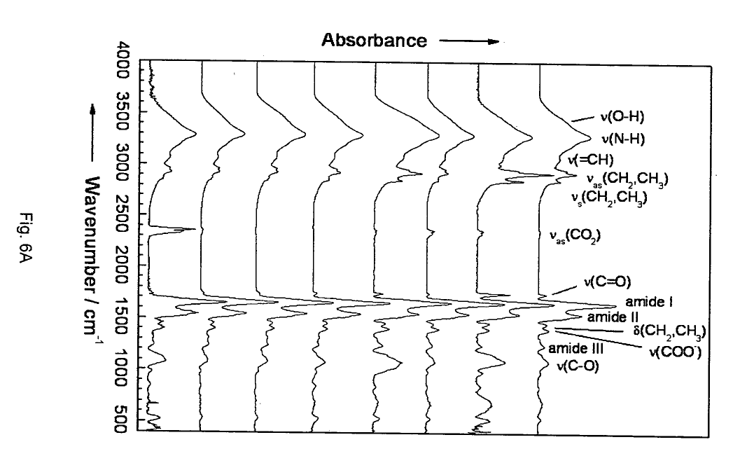

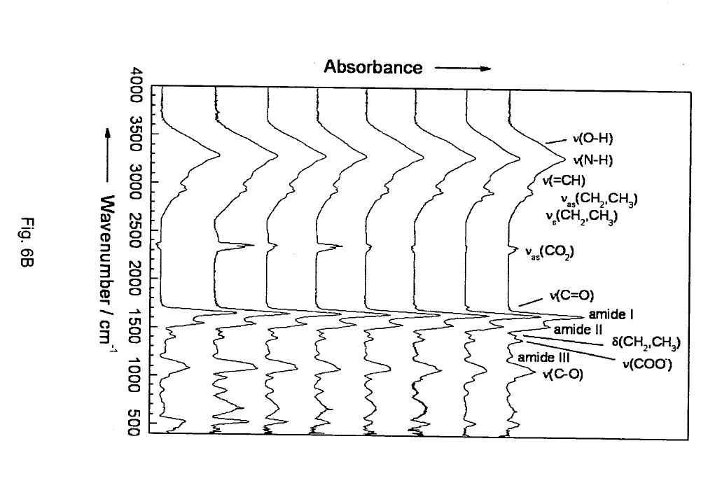

In FIGS. 6A and 6B, FT-IR spectra of plasma samples of healthy donors (FIG. 6A) and patients with thrombotic microangiopathy (FIG. 6B) are represented, illustrating an increased band in the region of 2900 cm⁻¹ and increased ester band in the region of 1740 cm¹ for healthy patients.

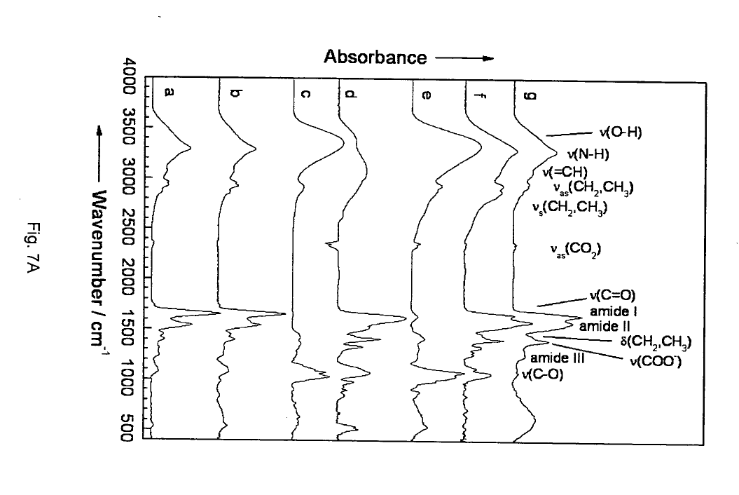

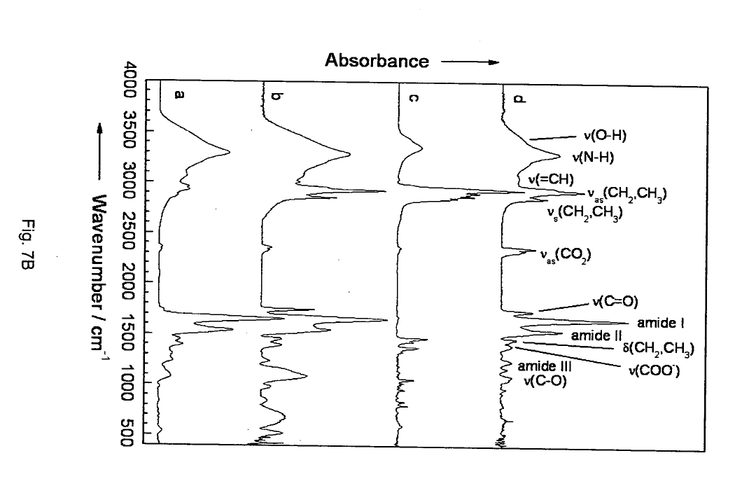

In order to identify the cause of the increased bands and to characterize the spectra of cryoprecipitate several plasma components such as proteins, glucose and lipids were analyzed. In FIG. 7A a FT-IR spectra of a human plasma sample of a healthy donor (a), cryosupernate (b), glucose (c), VWF-Factor VIII complex (d) clotting factor VIII (e), ultralarge VWF (f) and fibrinogen (g) are depicted. Furthermore high density lipoprotein and cholesterol was investigated to detect the cause of the increased bands. FIG. 7B shows FT-IR spectra of blood plasma of a healthy donor (a) (a), of a human plasma samples of a healthy donor showing an increased intensity at 1740 cm−1 and 2900 cm 1 (b), cholesterol (c) and high density lipoprotein from human plasma (d). Similar to Raman spectroscopy it was possible to assign this effect to lipids showing that also FT-IR spectroscopy is a feasible method for quality control of sample preparation.

Differences in the spectra of healthy donors and patients with TMA are not easily visualizable making a hierarchical cluster analysis necessary for distinguishing between them.

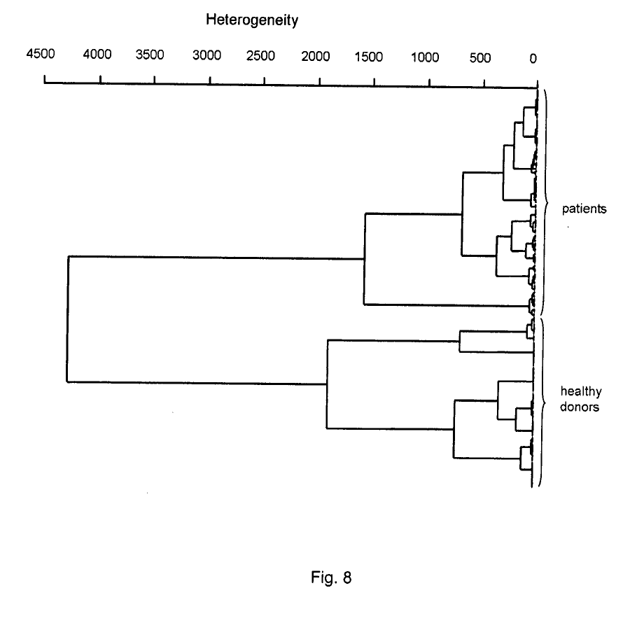

Only spectra without an increased peak at 1740 and 2900 cm⁻¹ were used for classification. The spectra were pretreated by baseline correction and vector normalization and the spectral range between 600 and 1800 cm⁻¹ and 2540-3680 cm⁻¹ was chosen for classification. The spectral distances between each spectrum were calculated with the method scaling to first range. Ward's technique was used to calculate the spectral distances between a newly created cluster and all of the other spectra or identified clusters. FIG. 8 shows the dendrogram of the resulted classification of the cryoprecipitated plasma samples based on 237 spectra of seven healthy controls and of ten different patient's samples The dendrogram shows a clear separation of healthy controls and patients without any misclassification. The asterisk indicates one wrong classified spectra of a healthy donor as a patient.

Definitions

“Diagnosis” in the context of the present invention refers to verifying whether an individual has suffered from an inflammatory associated coagulatory disturbance.

“Prognosis” in the context of the present invention refers to the prediction probability (in %) an individual will suffer from an inflammatory associated coagulatory disturbance.

“Therapy stratification” in the context of the present invention refers to assessing the appropriate therapeutic treatment for the inflammatory associated coagulatory disturbance which may occur or has occurred.

“Treatment monitoring” in the context of the present invention refers to controlling and, optionally, adjusting the therapeutic treatment of an individual.

“Therapeutic treatment” includes any treatment which may alter the pathophysiological state of an individual, and includes, for example, administering of pharmaceutical drugs as well as surgical treatment (e.g. by application of artificial surfaces like balloon dilatation, stenting).

“Spectroscopical analysis” in the context of the present invention refers to RAMAN spectroscopy, UV resonance Raman spectroscopy, surface enhanced Raman spectroscopy as well as Fourier transform (FT) infrared spectroscopy.

The present invention refers to thrombotic diseases such as inflammation associated microangiopathy, pregnancy associated microangiopathy, bone marrow transplatation associated microangiopathy, microangiopathy due to endocrine dysfunction and primary thrombotic microangiopathy such as TTP or HUS, which are caused or paralleled by an altered molecular and functional structure of VWF and/or changes in the activity of ADAMTS13.

### REFERENCES

1. Bone R C, Balk R A, Cerra F B, Dellinger R P, Fein A M, Knaus W A, Schein R M, Sibbald W J (1992) Definitions for sepsis and organ failure and guidelines for the use of innovative therapies in sepsis. The ACCP/SCCM Consensus Conference Committee. American College of Chest Physicians/Society of Critical Care Medicine. Chest 101: 1644-55.
2. Ackerman M H (1994) The systemic inflammatory response, sepsis, and multiple organ dysfunction: new definitions for an old problem. Crit Care Nurs Clin North Am 6: 243-50.
3. Kleinpell R M, Graves B T, Ackerman M H (2006) Incidence, pathogenesis, and management of sepsis: an overview. AACN Adv Crit Care 17: 385-93.
4. Balk R A (2004) Optimum treatment of severe sepsis and septic shock: evidence in support of the recommendations. Dis Mon 50:168-213.
5. Opal S M (2003) Interactions between coagulation and inflammation. Scand J Infect Dis 35: 545-54.
6. Kayal S, Jais J P, Aguini N, Chaudiere J, Labrousse J (1998) Elevated circulating E-selectin, intercellular adhesion molecule 1, and von Willebrand factor in patients with severe infection. Am J Respir Crit Care Med 157: 776-84.
7. Sadler J E (1998) Biochemistry and genetics of von Willebrand factor. Annu Rev Biochem 67: 395-424.
8. Tomokiyo K, Kamikubo Y, Hanada T, Araki T, Nakatomi Y, Ogata Y, Jung S M, Nakagaki T, Moroi M (2005) Von Willebrand factor accelerates platelet adhesion and thrombus formation on a collagen surface in platelet-reduced blood under flow conditions. Blood 105: 1078-84.
9. Nossent A Y, V VANM, N H VANT, Rosendaal F R, Bertina R M, J A VANM, Eikenboom H C (2006) von Willebrand factor and its propeptide: the influence of secretion and clearance on protein levels and the risk of venous thrombosis. J Thromb Haemost 4: 2556-62.
10. Furlan M (1996) Von Willebrand factor: molecular size and functional activity. Ann Hematol 72: 341-8.
11. Lopez J A, Dong J F (2005) Shear stress and the role of high molecular weight von Willebrand factor multimers in thrombus formation. Blood Coagul Fibrinolysis 16 Suppl 1: S11-6.
12. Tsai H M (2004) Molecular mechanisms in thrombotic thrombocytopenic purpura. Semin Thromb Hemost 30: 549-57.
13. Bonnefoy A, Daenens K, Feys H B, De Vos R, Vandervoort P, Vermylen J, Lawler J, Hoylaerts M F (2006) Thrombospondin-1 controls vascular platelet recruitment and thrombus adherence in mice by protecting (sub)endothelial VWF from cleavage by ADAMTS13. Blood 107: 955-64.
14. Vischer U M (2006) von Willebrand factor, endothelial dysfunction, and cardiovascular disease. J Thromb Haemost 4: 1186-93.
15. van Mourik J A, Boertjes R, Huisveld I A, Fijnvandraat K, Pajkrt D, van Genderen P J, Fijnheer R (1999) von Willebrand factor propeptide in vascular disorders: A tool to distinguish between acute and chronic endothelial cell perturbation. Blood 94: 179-85.
16. Moake J L (1998) von Willebrand factor in the pathophysiology of thrombotic thrombocytopenic purpura. Clin Lab Sci 11: 362-4.
17. Moake J L (1998) Moschcowitz, multimers, and metalloprotease. N Engl J Med 339:1629-31.
18. Gando S, Nanzaki S, Morimoto Y, Kobayashi S, Kemmotsu 0 (1999) Systemic activation of tissue-factor dependent coagulation pathway in evolving acute respiratory distress syndrome in patients with trauma and sepsis. J Trauma 47: 719-23.
19. Mannucci P M, Canciani M T, Forza I, Lussana F, Lattuada A, Rossi E (2001) Changes in health and disease of the metalloprotease that cleaves von Willebrand factor. Blood 98: 2730-5.
20. Nguyen T C, Liu A, Liu L, Ball C, Choi H, May W S, Aboulfatova K, Bergeron A L, Dong J F (2007) Acquired ADAMTS-13 deficiency in pediatric patients with severe sepsis. Haematologica 92: 121-4.
21. Ono T, Mimuro J, Madoiwa S, Soejima K, Kashiwakura Y, Ishiwata A, Takano K, Ohmori T, Sakata Y (2006) Severe secondary deficiency of von Willebrand factor-cleaving protease (ADAMTS13) in patients with sepsis-induced disseminated intravascular coagulation: its correlation with development of renal failure. Blood 107: 528-34.
22. Barington K A, Kaersgaard P (1999) A very-high-purity von Willebrand factor preparation containing high-molecular-weight multimers. Vox Sang 76: 85-9.
23. Schneppenheim R, Budde U, Oyen F, Angerhaus D, Aumann V, Drewke E, Hassenpflug W, Haberle J, Kentouche K, Kohne E, Kurnik K, Mueller-Wiefel D, Obser T, Santer R, Sykora K W (2003) von Willebrand factor cleaving protease and ADAMTS13 mutations in childhood TTP. Blood 101: 1845-50.
24. Gerritsen H E, Turecek P L, Schwarz H P, Lammle B, Furlan M (1999) Assay of von Willebrand factor (vWF)-cleaving protease based on decreased collagen binding affinity of degraded vWF: a tool for the diagnosis of thrombotic thrombocytopenic purpura (TTP). Thromb Haemost 82: 1386-9.
25. Ruggeri Z M, Zimmerman T S (1981) The complex multimeric composition of factor VIII/von Willebrand factor. Blood 57: 1140-3.
26. Schneppenheim R, Plendl H, Budde U (1988) Luminography—an alternative assay for detection of von Willebrand factor multimers. Thromb Haemost 60: 133-6.
27. Budde U, Schneppenheim R, Plendl H, Dent J, Ruggeri Z M, Zimmerman T S (1990) Luminographic detection of von Willebrand factor multimers in agarose gels and on nitrocellulose membranes. Thromb Haemost 63: 312-5.

1. A method of analyzing the cleavage profile and size distribution of von Willebrand factor (VWF) multimers, comprising: providing a sample medium or human body fluids comprising a plurality of VWF multimers of different size; enrichment or purification of VWF by cryoprecipitation or chromatography from said sample medium; exposing said separated preparation of VWF multimers to a light source to produce signals obtained by vibrational spectroscopy; detecting said signals; transformation by mathematical algorithms; generation of patterns based on computing of data of original resonance spectra and determining the cleavage profile and the size distribution of said separated VWF multimers by chemometrics and acquisition of a databank obtained from healthy individuals identifying subjects at risk of developing thrombotic disease and/or identifying subjects at risk of developing infection, inflammation in particular sepsis and/or coagulopathy.

2. The method of claim 1 wherein analysis of the cleavage profile and size distribution of von Willebrand factor (VWF) multimers is used for therapeutically monitoring and decision making for these subjects.

3. The method of claim 1 wherein analysis of the cleavage profile and size distribution of von Willebrand factor (VWF) multimers is used for screening of subjects at risk of developing thrombotic disease, infection, inflammation in particular sepsis and/or coagulopathy.

4. The methods of claim 1 wherein analysis of the cleavage profile and size distribution of von Willebrand Factor (VWF) multimers from said samples is used for comparing spectra corresponding to a set of VWF abnormalities and/or disease for diagnostic approaches.

5. The methods of claim 1 wherein analysis of the cleavage profile and size distribution of von Willebrand Factor (VWF) multimers from said samples is used for comparing spectra detecting the VWF-degrading activity of proteolytic enzymes, in particular of the ADAMTS13 protease.

6. The methods of claims 1-5 wherein analysis of the cleavage profile and size distribution of von Willebrand Factor (VWF) multimers from said samples is used for early diagnosis and/or differential diagnosis and/or monitoring of the disease.

7. Methods of claims 1-6 wherein analysis of the cleavage profile and size distribution of von Willebrand Factor (VWF) multimers from said samples is used for correlational analysis with non-spectroscopic data.

8. Methods of claims 1-7 wherein the plurality of signals of said sample is analysed by the use of non-supervised classification analysis, in particular comprising hierarchical clustering and principal component analysis.

9. Methods of claims 1-7 wherein the plurality of signals of said samples is analysed by the use of supervised classification analysis, in particular comprising K-nearest neighbour analysis, nearest mean classifier, linear discrimination analysis, artificial neural networks, as well as support vector machines.

10. Methods of claims 1-9 wherein analysis of the cleavage profile and size distribution of von Willebrand Factor (VWF) multimers from said samples is used for monitoring the administered therapeutically effective amount of a recombinant ADAMTS13 or genetic material comprising an ADAMTS13 gene or mutant or variant thereof.

11. Methods of claims 1-10 wherein analysis of the cleavage profile and size distribution of von Willebrand Factor (VWF) multimers from said samples is used for monitoring the administered therapeutically effective amount of a therapeutically effective amount of ADAMTS13 protease such that the symptoms of the disease are alleviated, wherein the ADAMTS13 protease is selected from the group consisting: recombinant ADAMTS13; synthetic ADAMTS13; mutants, variants, fragments, and fusions of recombinant ADAMTS13; and mutants, variants, fragments, and fusions of synthetic ADAMTS13.

12. Methods of claims 1-11 wherein analysis of the cleavage profile and size distribution of von Willebrand Factor (VWF) multimers from said samples is used for monitoring the efficiency of drugs or drug candidates for the treatment of thrombotic diseases, infection, inflammation and/or coagulopathy such that the application of the drug or the drug candidates influence the ADAMTS13 activity and/or the cleavage profile and size distribution of VWF multimers whereas alteration of ADAMTS13 activity and/or the cleavage profile and the size distribution of VWF after application of the drug or the drug candidate is used for indicating the efficiency of the drug or the drug candidate.

13. Methods of claims 1-11 wherein analysis of the cleavage profile and size distribution of von Willebrand Factor (VWF) multimers from said samples is used for monitoring the treatment of diseases or altering physiological states characterized by decreased VWF-cleaving protease activity, and/or pathologic platelet aggregation whereas alteration of ADAMTS13 activity and/or the cleavage profile and the size distribution of VWF and/or parameters of platelet aggregation after application of the drug or the drug candidate is used for indicating the efficiency of the drug or the drug candidate.
